(19) United States
(12) Patent Application Publication
(10) Pub. No.: US 2009/0053799 A1
(43) Pub. Date: Feb. 26, 2009

(54) TRAPPING MAGNETIC SORTING SYSTEM FOR TARGET SPECIES

(75) Inventors: David A. Chang-Yen, Oxnard, CA (US); Jafar Darabi, Goleta, CA (US); Yanting Zhang, Goleta, CA (US); Hyongsok T. Soh, Santa Barbara, CA (US); Paul Pagano, Santa Barbara, CA (US)

Correspondence Address: Weaver Austin Villeneuve & Sampson LLP, P.O. BOX 70250, OAKLAND, CA, 94612-0250, US

(73) Assignee: Cynvenio Biosystems, LLC, Santa Barbara, CA (US)

(21) Appl. No.: 12/197,169

(22) Filed: Aug. 22, 2008

Related U.S. Application Data
(60) Provisional application No. 60/966,092, filed on Aug. 23, 2007.
(60) Provisional application No. 61/037,994, filed on Mar. 19, 2008.

(51) Int. Cl. C12M 1/00

(52) U.S. Cl. 435/287.2; 435/306.1

(57) ABSTRACT

A system for sorting and trapping magnetic target species includes a microfluidic trapping module designed to receive and then temporarily hold magnetic particles in place within the module. The magnetic particles flowing into the module are trapped there while the other sample components (non-magnetic) continuously flow through and out of the station, thereby separating and concentrating the species captured on the magnetic particles. The magnetic particles and/or their payloads may be released and separately collected at an outlet after the sample passes through the trapping module.

### CROSS REFERENCE TO RELATED APPLICATIONS

This application claims priority to U.S. Provisional Patent Application No. 60/966,092, filed Aug. 23, 2007, titled Trapping Magnetic Cell Sorting System, and to U.S. Provisional Patent Application No. 61/037,994, filed Mar. 19, 2008, titled Trapping Magnetic Cell Sorting System, the disclosures of both applications are incorporated herein by reference in their entireties and for all purposes.

### BACKGROUND

Sorting chemical or biological species based on their surface markers is an important capability in biology and medicine. Magnetic Activated Cell Sorting (MACS) is sometimes used as a cell sorting technique because it allows the rapid selection of a large number of target cells. The applications of MACS span a broad spectrum, ranging from protein purification to cell based therapies. Typically, target cells are labeled with one or more superparamagnetic particles that are conjugated to a molecular recognition element (e.g. a monoclonal antibody) which recognizes a particular cell surface marker.

Application of MACS has frequently been limited to pre-enrichment before fluorescence-based cytometry. Nevertheless, due to its high throughput compared to other methods such as Fluorescence Activated Cell Sorting (FACS), MACS is still a competitive technology.

In order to achieve high throughput and high recovery of the rare cells (or other target components), improvements on existing MACS systems are needed.

### SUMMARY

In various embodiments, a fluidic sorting module is designed to receive and then temporarily hold magnetic particles in place within the module. Later, the particles and/or their captured species are released, collected, and/or further processed. In such embodiments, the magnetic particles flowing into the sorting station are trapped there while the other sample components (non-magnetic) continuously flow through and out of the station, thereby separating and concentrating the species captured on the magnetic particles. Only after the non-magnetic sample components have flowed out of the sorting chamber are the magnetic particles and/or their payloads released and separately collected at an outlet of the sorting station.

One aspect of the present invention pertains to a microfluidic sorting device for sorting a sample into substantially target and substantially non-target species. The device includes a trapping module, a pre-processing and/or a post-processing station. The trapping module includes a channel having two opposing walls, one of which includes a magnetic field gradient generating structure for exerting a magnetic force on a sample to capture, at least temporarily, magnetic particles in the sample. The pre-processing and/or post-processing station is integrated on the microfluidic sorting device with trapping module.

The flow channel in the trapping module may include opposing walls, an opaque wall and a transparent wall or two opaque walls. The magnetic field gradient generating structure may be on the opaque wall. The magnetic field gradient generating structure may be a ferromagnetic structure laid out in a pattern or randomly on its opposing wall. The pattern may be parallel lines, an orthogonal grid, a rectangular array of regular or irregular geometric shapes, and combinations thereof. The ferromagnetic structure may be nickel, vanadium permedur, permalloy, or other ferromagnetic material.

The flow channel includes an inlet region, a trapping region, and an outlet region. The depth of the channel in the trapping region may be larger than the depth of inlet and outlet regions, preferably about 2 times to that of inlet or outlet regions. A plurality of independent flow channels may be used. These flow channels may share a buffer manifold and may be configured to process from the same sample or different samples. In certain embodiments, multiple trapping regions may be used in the same flow channel.

The microfluidic sorting device may include an external magnetic source on one side of the trapping module, preferably the side of the magnetic field gradient generating structure, for generating a magnetic field. In certain embodiments, the magnetic field is progressively applied to a trapping station to oppose the fluid flow within said trapping station to thereby cease movement as the trapping region is gradually addressed by said magnetic field. In other words, the magnetic field is shifted so as to produce a time varying magnetic field in the trapping region, thereby inducing a desired magnetic particle motion.

This may serve to spread the magnetic bead bound target particles over the trapping region in a uniform manner. This may facilitate, inter alia, post-separation operations, such as bead release by allowing a release reagent to efficiently access magnetic bead-bound target species. The progressive application of the magnetic may be accomplished by moving the external magnetic source with respect to the trapping module. The external source may be one or more permanent magnets, one or more electromagnets, or a combination of these.

The pre-processing station may include a labeling station for labeling a species in the sample with magnetic particles having an affinity for the labeled species. Note that the labeled species may be the target species or the non-target species. If the non-target species labeled, the target species concentration in the sample passing through the trapping module is increased by trapping the non-target species. If the target species is labeled, the target species are trapped in the trapping module and later collected.

In certain embodiments, at least one of pre-processing station or the post-processing station includes an enrichment module for increasing a concentration of a target species in a sample passing through the sorting device, a reaction module, a detection module, and a lysis module for lysing cells, disrupting viral protein coats, or otherwise releasing components of small living systems. The trapping module may be designed or configured to perform an operation other than trapping, including genomic analysis, detection and/or amplification scheme for DNA or RNA oligomers, gene expression, enzymatic activity assays, receptor binding assays, and ELISA assays.

Another aspect of the present invention pertains to a microfluidic sorting device for sorting a sample into substantially target and substantially non-target species. The device includes a trapping module, a pre-processing and/or a post-processing station as described above, an external source on one side of the trapping module for generating a magnetic field, and a mechanism to varying the magnetic field over time. The trapping module includes a channel having two opposing walls and no magnetic field gradient generating structure. In one embodiment, the side of the trapping module having the external magnetic source is opaque.

Yet another aspect of the present invention pertains to a method for sorting a sample in a microfluidic sorting device that includes a trapping module and a magnetic field gradient generating structure on only one side of two opposing walls of the trapping module. The method includes flowing a sample into the trapping module, generating a magnetic field gradient in the trapping module, and trapping magnetic particles in the trapping module. The sample includes magnetic particles with molecular recognition elements thereon, a target species, and a non-target species. An external magnetic field is exerted from only one side of the module to the magnetic field gradient generating structure. The magnetic particles are trapped proximate to the magnetic field gradient generating structure.

The sorting method may also include moving an external source of the magnetic field with respect to the trapping module while the magnetic particles flow through the module in the fluid medium to thereby trap magnetic particles in a substantially uniform fashion. The magnetic field may be progressively applied to a trapping station to oppose the fluid flow within said trapping station to thereby cease movement as the trapping region is gradually addressed by said magnetic field. In other words, the magnetic field is shifted so as to produce a time varying magnetic field in the trapping region, thereby inducing a desired magnetic particle motion to spread the magnetic bead bound target particles over the trapping region in a uniform manner.

In certain embodiments, the sorting method may include releasing the magnetic particles from one section of the magnetic field gradient generating structure to release any trapped non-magnetic particles and the trapping the magnetic particles in another section of the magnetic field gradient generating structure or in another magnetic field gradient generating structure. The sorting method may also include labeling the target species in the sample with magnetic particles having an affinity for the target species or labeling the non-target species in the sample with magnetic particles having an affinity for the non-target species. The sorting method may also include detecting the target species in a microarray, lysing the target species, reacting the target species, or imaging the trapped target species. The reacting operation may include amplifying, sequencing, hybridizing, labeling, crosslinking or culturing the target species. Note that these pre-processing and post-processing operations may occur in the trapping module or in another module on the same microfluidic device.

These and other features and embodiments of the invention will be described in more detail below with reference to the associated drawings.

### BRIEF DESCRIPTION OF THE DRAWINGS

FIG. 1A shows a system that employs disposable fluidics chips or cartridges.

FIG. 1B is a process flow diagram showing a method of using the system of FIG. 1A.

FIGS. 1C and 1D illustrates a top view and a side view of a magnetic trapping module in accordance with certain embodiments.

FIG. 2 is a process flow diagram of a method of sorting a sample in accordance with various embodiments of the present invention.

FIG. 3 depicts an embodiment in which a magnetic field producer moves over one surface of the trapping region during the passage of magnetic particles through the trapping region.

FIGS. 4A to 4C present an example of a staged capture and release trapping system.

FIG. 5 depicts a fluidics input for a sample well and a bead release reagent well.

FIGS. 6A to 6H show various structures of a magnetic trap disposed in a fluidics device for post-capture treatment of target species.

FIG. 7 presents examples of non-magnetic capture features fabricated among a soft-magnetic (e.g., nickel) pattern.

FIGS. 8A to 8C depict examples of random array of ferromagnetic structures.

FIGS. 9A and 9B depict side views of fluidic channel embodiments with large depths at the trapping region.

FIG. 9C depicts an embodiment with parallel fluidic channels sharing a common buffer manifold.

FIG. 9D depicts a fluidic channel with two trapping regions.

### DESCRIPTION OF CERTAIN EMBODIMENTS

Introduction and Context

Magnetic Activated Cell Sorting (MACS) systems are capable of high-purity selection of the labeled cells or other sample components. In certain embodiments these systems operate in a “trapping mode” where the non-target and target species are sequentially eluted after the application of the external magnetic field. In other words, the species attached to magnetic particles are held in place while the unattached species are eluted. Then, after this first elution step is completed, the species that are attached magnetic field and were prevented from being eluted are freed in some manner such that they can be eluted and recovered.

In accordance with some embodiments, a trapping module of a MACS system includes a channel through which a sample, including species attached and not attached to magnetic particles flow. One side of the channel includes a magnetic field gradient generating structure that generates a magnetic field gradient with the application of a magnetic field from an external source. This magnetic field gradient attracts and captures magnetic particles along with the attached species. After the sample flows through the trapping module, the captured particles may be released by changing the applied magnetic field or by cleaving the link between the magnetic particles and the attached species.

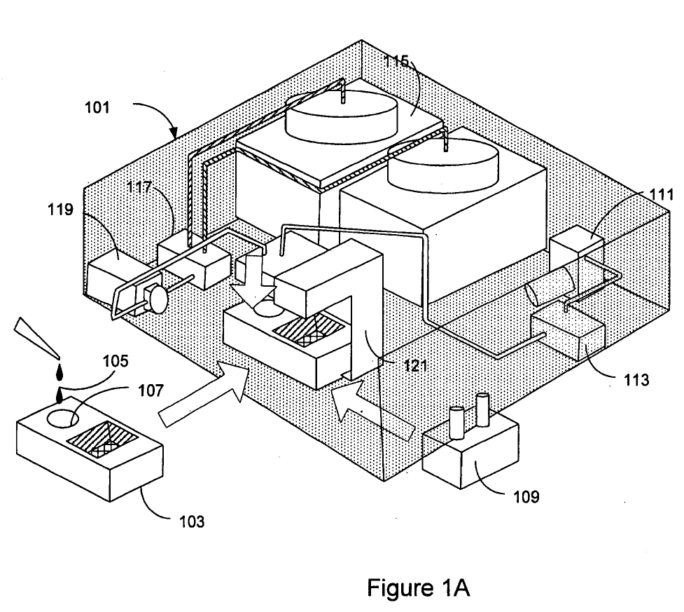

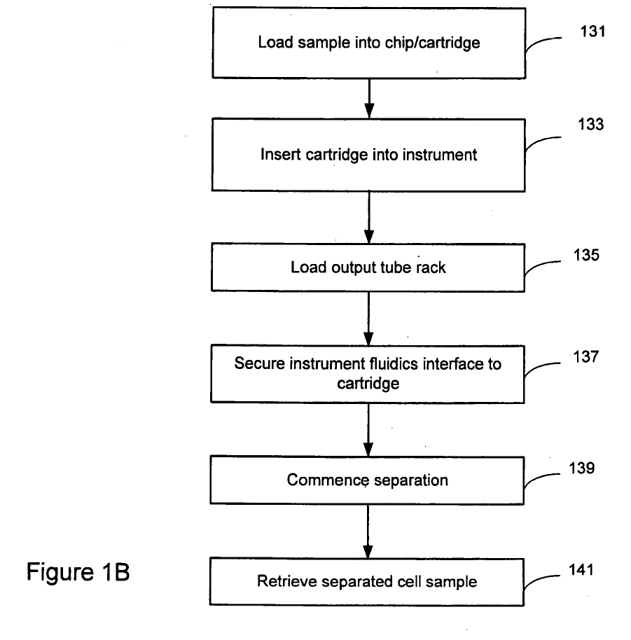

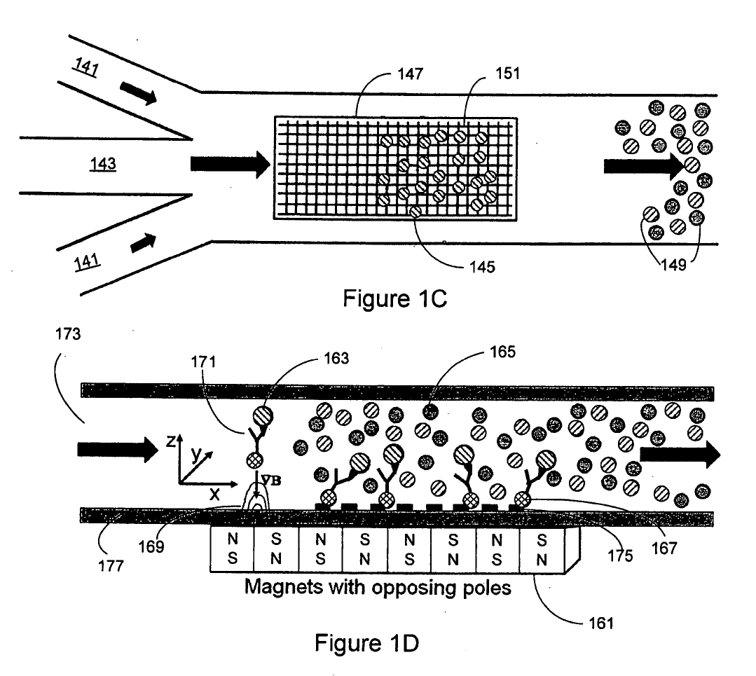

For context an example of a trapping-type magnetic separation system will now be described. FIGS. 1A and 1C illustrate magnetic sorting modules and systems in accordance with certain embodiments. FIG. 1A shows a system 101 that employs disposable fluidics chips or cartridges 103. Each chip or cartridge houses fluidics elements that include a magnetic trapping module. In one mode of operation (positive selection), a sample 105 such as a small quantity of blood is provided to a receiving port 107 of the cartridge and then the cartridge with sample in tow is inserted into a processing and analysis instrument 121. Within the chip, the magnetic particles and the target species (if any) from the sample are sorted and concentrated at the magnetic trapping module. After sample has been processed in this manner, trapped species may be released and collected in output tubes 109. This may be accomplished by various means including reducing or eliminating the external magnetic field applied to the trapping module or applying a reagent that releases captured species from magnetic particles. Alternatively, or in conjunction, the hydrodynamic force exerted on the magnetic particles may be increased. In the depicted embodiment, a chassis houses the system components including a pressure system (including a syringe pump 111 and a pressure controller 113) that provides the principal driving force for flowing sample through the trapping module. Of course, other designs may be employed using alternative driving forces such as a continuous pump or a pneumatic system. Buffer from buffer reservoirs 115 is also provided to the cartridge under the controlled by a buffer pump 119 and a flow control module 117.

In FIG. 1B, an example processing sequence is shown. Specifically, the process begins by loading a sample onto the chip or cartridge before inserting into the instrument, shown as operation 131. Then, in operation 133, the chip is inserted into the instrument to align the external magnet(s), fluidics couplings, and associated apparatus. Thereafter, a collection tube or tubes is also loaded into the instrument (operation 135). Note that in some embodiments the order of loading to the instrument can be varied. Next, a fluidics interface is secured to the chip to ensure leakproof delivery of sample and buffer to the chip in operation 137. Finally, the instrument commences the separation process (139) and separated cell sample may be retrieved by unloading the collection tube (141).

FIGS. 1C and 1D show top and side views of a trapping module in accordance with one embodiment. In a specific example, the depicted trapping module is implemented in a disposable cartridge as shown in FIG. 1A. In FIG. 1C, a top view of the magnetic trapping module is shown to include a central sample inlet 143, and two buffer inlets 141 straddling the sample inlet 143. Buffer delivered from the buffer inlets may prevent contents of the sample from becoming entrained along the edge of the trapping module, and help to stabilize the pressure as well as the flow streams. As shown, a trapping region 147, which in this embodiment includes a ferromagnetic pattern 151 is formed on a bottom wall of a flow channel. The channel wall on which the pattern is formed may be transparent, semi-transparent or opaque.

As shown, target species 145 are captured on the trapping region. The remaining uncaptured cells 149 (or other species) and debris provided with the sample are washed clear of the trapping region because they are not affixed to magnetic particles.

It should be noted that positive or negative trapping schemes may be employed. In a positive trapping scheme as shown in FIGS. 1C and 1D, target species, e.g., 145 and 163, are linked via a linker 171 to the surfaces of the magnetic particles 167 and are thereafter trapped together with the magnetic particle in the trapping region. This effectively purifies and concentrates the target species. In negative trapping embodiments, non target species (rather than target species) are labeled with magnetic particles. Thus, the unlabeled target species continuously flow through the trapping module, while the labeled non-target species are trapped in the trapping region and removed from suspension. This approach purifies the target species, but does so without concentrating them.

A side view of the trapping region in action is depicted in FIG. 1D. As shown there, ferromagnetic structures 175 are formed on the inside surface of a lower wall 177 of a flow channel 173. These serve as a magnetic field gradient generating (MFG) structures (described in more detail below). An external magnetic field 169 is typically used as the driving force for trapping the magnetic particles flowing through the fluid medium. The MFG structures 175 may shape the external magnetic field in order to create locally high magnetic field gradients to assist capturing flowing magnetic particles 167. In the depicted embodiment of FIG. 1D, the external magnetic field is provided by an array of permanent magnets 161 of alternating polarity. More generally, the external magnetic field may be produced by one or more permanent magnets and/or electromagnets. In some embodiments, a collection of magnets such as those shown in FIG. 1D are moveable, individually or as a unit, in order to dynamically vary the magnetic field applied to the trapping region.

In certain embodiments, the magnetic field is controlled using an electromagnet. In other embodiments, permanent magnets may be used, which are mechanically movable into and out of proximity with the sorting station such that the magnetic field gradient in the sorting region can be locally increased and decreased to facilitate sequential capture and release of the magnetic particles. In some cases using an electromagnet, the magnetic field is controlled so that a strong field gradient is produced early in the process (during capture of the magnetic particles) and then reduced or removed later in the process (during release of the particles).

As shown in the example of FIG. 1D, the magnetic particles are coated with one or more molecular recognition elements 171 (e.g., antibodies) specific for a marker of a target cell 163 or other species to be captured. Thus, one or more magnetic particles 167, along with a bound cell or other target species 163, flow as a combined unit into the trapping module. For large target species having many exposed binding moieties (e.g., mammalian cells), it will be common to have multiple magnetic particles affixed.

In some embodiments, the trapping region is relatively thin but may be quite wide to provide relatively high throughput. In other words, the cross-sectional area of the channel itself is relatively large while the height or depth of the channel is quite thin. The thinness of the channel may be defined by the effective reach of the magnetic field which is used to attract the magnetic particles flowing through the trapping region in the fluid medium.

Various details of fluidics systems suitable for use with this invention are discussed in other contexts in the description of flow modules in U.S. patent application Ser. No. 11/583,989 filed Oct. 18, 2006, which is incorporated herein by reference for all purposes. Examples of such details include buffer composition, magnetic particle features, external magnet features, ferromagnetic materials for MFGs, flow conditions, sample types, integration with other modules, control systems for fluidics and magnetic elements, binding mechanisms between target species and magnetic particles, etc. Generally, in a magnetic trapping module the applied external magnetic field will be relatively higher (considering the overall design of the module) than that employed in a continuous flow magnetic flow sorter of the type described in U.S. patent application Ser. No. 11/583,989. In any event, the magnetic force exerted on target species should be sufficiently greater than the hydrodynamic drag force in order to ensure that the target species (coupled to magnetic particles) are captured and held in place against the flowing fluid.

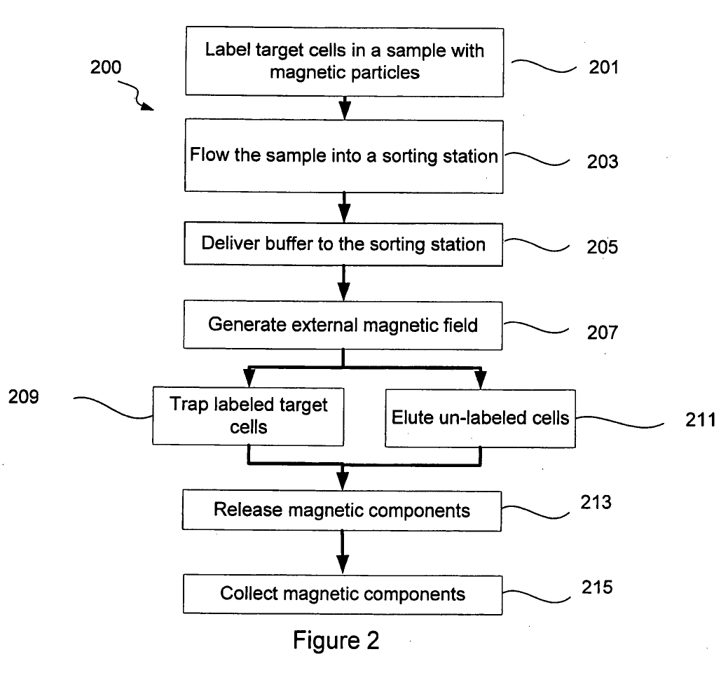

In a typical positive selection example as shown in FIG. 2, the magnetic trapping process 200 proceeds as follows. First, a sample such as a biological specimen potentially containing the target cells are labeled with small magnetic particles coated with a capture moiety (e.g., an antibody) specific for the surface marker of the target cell in operation 201. This labeling process may take place on or off the microfluidic sorting device. After this labeling, the sample is flowed into the sorting station (comprising a trapping region) with or without concurrently flowing buffer solution in operation 203. Buffer may be delivered through one or more inlets and sample through one or more others. The sorting station is energized with an external magnetic field in operation 207 to hold the magnetically labeled target cells or other species in place against the hydrodynamic drag force exerted by the flowing fluid in operation 209. This occurs while continuously eluting the un-labeled non-target species in operation 211. As explained above, the magnetic field is typically applied by an external magnet positioned proximate the sorting station. After most, or all, of the sample solution has flowed clear of the sorting station, the magnetic components may be released in operation 213 by any of a number of different mechanisms including some that involve modifying the magnetic field gradient and/or increasing the hydrodynamic force. For example, the magnetic field in the chamber may be reduced, removed, or reoriented and concurrently the sample inlet flow is replaced with release agent (for releasing the captured species) and/or buffer flow. Ultimately the previously immobilized magnetic components, or just their captured species (now purified), flow out of the chamber in a buffer solution. The purified sample component comprising the target species may then be collected at an outlet of the sorting chamber in operation 215, which, in some configurations may be located directly downstream from the trapping chamber.

A capture and release protocol is particularly advantageous when using large target species such as mammalian cells that respond strongly to hydrodynamic forces and relatively weakly to magnetic forces (possibly because only one or a small number of magnetic particles attached to the cell are influenced by the magnetic field gradient generating structures). The capture and release protocol may also be beneficial when using relatively small target species such as viruses which have a tendency to become entrained in a boundary layer of a flow field within a microfluidic device.

There are various features and advantages of using trapping type sorting modules. Among these are the following.

1. The target species can be greatly concentrated because only a small elution volume is used to release the trapped target species. Over time, target species from a low concentration sample are extracted and held fixed until the entire sample is processed. Then the captured species are released in a relatively small volume of carrier medium, thereby producing a high purity, high concentration solution or suspension.

2. The physical dimensions of the sorter can be relatively large because it may employ relatively large magnetic fields, influencing magnetic particles over relatively large distances in a sorting module. As an example, the flow channel height may be 20 micrometers or larger. This allows for relatively high throughput (e.g., at least about 10 ml/hour, or 50 ml/hour, or 100 ml/hour, or 1 litre/hour).

3. A monolayer (or sub-monolayer) of captured species can be produced. Alternatively, a layer consisting of only a few sub-layers (e.g., a bilayer or trilayer) can be produced. In either case, large “clumps” which might constrict the flow passage or otherwise interfered with trapping can be avoided. This is possible because the external field can be dynamically controlled as described below. Alternatively, or in addition, MFGs can be employed to limit application of very strong magnetic forces on magnetic particles over only small distances. Limiting captured species to a monolayer has various advantages. One of these is in providing an unobstructed flow path above the monolayer. Hence it is unlikely that non-target species will become entrained in a mass of target species while flowing through the trapping module. Another advantage resides in the ability to image distinct species of monolayer at a well defined depth of focus.

4. An array of external magnets may be employed (see e.g., FIG. 1D). This allows fine tuning of the magnetic field over the domain of the sorting module. In some embodiments, the array of magnets employs alternating polarity magnets as shown in FIG. 1D, although this is not necessary. In some embodiments, only two magnets are employed (both disposed below the MFGs).

5. The dimensions and shape of the flow channel in the sorting module can be varied over the flow path in order to control hydrodynamic forces acting on the magnetic particles (and associated target species). In this way, the balance of magnetophoretic and hydrodynamic forces can be tailored to yield a high performance separation.

It should be understood that embodiments of the invention are not limited to analysis of biological or even organic materials, but extend to non-biological and inorganic materials. Thus, the apparatus and methods described above can be used to screen, analyze or otherwise process a wide range of biological and non-biological substances in liquids. The target and/or non-target species may comprise small or large chemical entities of natural or synthetic origin such as chemical compounds, supermolecular assemblies, organelles, fragments, glasses, ceramics, etc. In certain embodiments, they are monomers, oligomers, and/or polymers having any degree of branching. They may be expressed on a cell or virus or they may be independent entities. They may also be complete cells or viruses themselves.

The external magnet (or a system of magnets) may be variably positioned during capture of the magnetic particles, and as explained may be a permanent magnet or electromagnet, or multiples of either of these or combinations of permanent and electromagnets.

The magnetic capture particles employed in separations of this invention may take many different forms. In certain embodiments, they are superparamagnetic particles or nanoparticles, although in some cases they may be ferromagnetic or paramagnetic. As a general proposition, the magnetic particles should be chosen to have a size, mass, and susceptibility that allow them to be easily diverted from the direction of fluid flow when exposed to a magnetic field in microfluidic device (balancing hydrodynamic and magnetic effects). In certain embodiments, the particles do not retain magnetism when the field is removed. In a typical example, the magnetic particles comprise iron oxide (Fe₂O₃ and/or Fe₃O₄) with diameters ranging from about 10 nanometers to about 100 micrometers. However, embodiments are contemplated in which even larger magnetic particles are used.

The magnetic particles may be coated with a material rendering them compatible with the fluidics environment and allowing coupling to particular target components. Examples of coatings include polymer shells, glasses, ceramics, gels, etc. In certain embodiments, the coatings are themselves coated with a material that facilitates coupling or physical association with targets. For example, a polymer coating on a micromagnetic particle may be coated with an antibody, nucleic acid sequence, avidin, or biotin.

One class of magnetic particles is the nanoparticles such as those available from Miltenyi Biotec Corporation of Bergisch Gladbach, Germany. These are relatively small particles made from coated single-domain iron oxide particles, typically in the range of about 10 to 100 nanometers diameter. They are coupled to specific antibodies, nucleic acids, proteins, etc. Magnetic particles of another type are made from magnetic nanoparticles embedded in a polymer matrix such as polystyrene. These are typically smooth and generally spherical having diameters of about 1 to 5 micrometers. Suitable beads are available from Invitrogen Corporation, Carlsbad, Calif. These beads are also coupled to specific antibodies, nucleic acids, proteins, etc.

Producing Even Distributions of Particles in Trapping Regions

Various techniques and device designs may be employed to facilitate even distribution of trapped magnetic particles over the surface of a trapping region. In some cases, the layer trapped particles is effectively a monolayer of magnetic particles on the trapping region, although sub-monolayers as well as bilayers and the like may be produced depending upon the area of the trapping region and the quantity of sample to be processed.

One approach involves carefully designing an arrangement of magnetic field gradient shaping elements in the trapping region. In certain embodiments, the ferromagnetic pattern spacing forming the magnetic field gradient generating structure is reduced in the downstream direction. In other words, the design of the grid may be varied as a function of position. This approach promotes a magnetic particle trajectory in which particles entering the trapping module are initially drawn down toward the substrate and then trapped on the magnetic field gradient generating structures in a monolayer. Very strong magnetic forces over only small distances are generated on the structures to trap the particles. The distance into the flow stream over which the magnetic forces are strong may be controlled to be the length of one monolayer of magnetic particles and labeled target particle. In some cases, bilayers and the like may be produced by self-magnetization of captured magnetic particles that then acts as capture structures for subsequent magnetic particles flowing through the trapping module.

Another approach involves dynamically varying the external magnetic field applied to the trapping region during flow of the magnetic particles. This may involve, for example, progressive insertion of a magnetic field over the trapping region during the trapping operation.

These and other approaches have the advantage of reducing or preventing build up of magnetic particles at the leading edge or elsewhere in the trapping region. Generally, a build up has been observed to occur where the magnetic field is strongest, typically at the edge of a permanent magnet used to apply the external magnetic field. As should be clear, such build up can result in under utilization of the trapping region (portions of the trapping region where the magnetic field strength is not great might not capture many or any of the magnetic particles). Further, the clump or pile up of magnetic particles may actually block passage of further magnetic particles to the down stream portions of the trapping region. It may also capture unbound species from the sample and thereby reduce purity of the captured components of the sample.

By using a carefully designed MFG structure and layout and/or a dynamically varying magnetic field, one can produce a relatively evenly dispersed layer of the magnetic particles captured over the trapping region. Other techniques for accomplishing the same or a similar result involve a plurality of thin permanent magnets of alternating polarity arranged side by side along the trapping region (axial direction) or in a checkerboard pattern of alternating polarity (both axial and lateral directions).

A relatively uniform distribution of magnetic particles in the trapping region may be useful during post-separation operations such as bead release. The release agent will fill the entire the channel and the uniform spreading of magnetic bound target particles will allow greater access to the magnetic bead bound target particles by the release agent.

In accordance with some embodiments of this invention, the position of greatest magnetic field strength is gradually moved over the trapping region during the period of time when particles are flowing into the channel. The direction of movement of the magnetic field during trapping may be, in one embodiment, from a down stream position to an up stream position within the trapping region. In other words, the direction of movement of the magnetic field is opposite that of the direction of the fluid flow in the trapping region. Such embodiments may involve, for example, moving a permanent magnet in a direction from a downstream position to an upstream position underneath the base of a flow channel, particularly the region of the channel comprising the trapping region. Thus, as magnetic particles first enter the trapping region, the leading edge of the permanent magnet is positioned just beyond the downstream edge of the trapping region. Thereafter, as the magnetic particles begin to flow into the trapping region, the leading edge of the permanent magnet is gradually moved upstream and ultimately comes to rest at or near the upstream boundary of the trapping region. In certain embodiments, it reaches its position at about the time when the magnetic particles cease flowing into the trapping region.

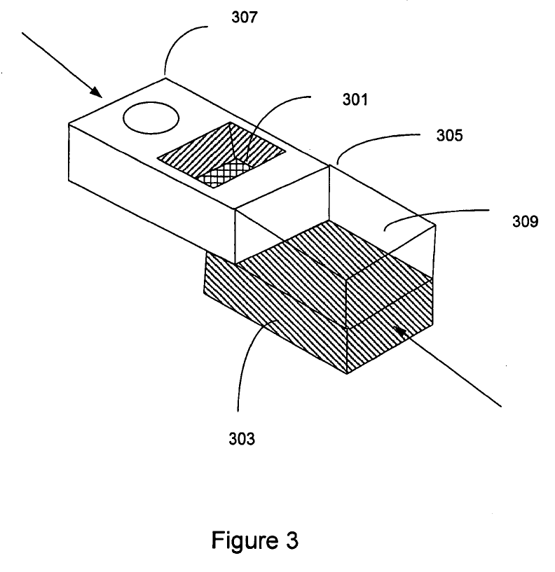

In an alternative embodiment, the external magnet moves from the upstream to the downstream positions of the trapping region during capture of the magnetic particles. In other words, the external magnet moves in the same direction as the fluid flow. In this embodiment, as in the previously described embodiment, the duration of the movement of the external magnet should correspond, at least roughly, to the period of time during which magnetic particles are flowing through the trapping region. As explained below with reference to FIG. 3, one specific embodiment employs a downstream movement of a magnet to sequentially capture and release and capture and release . . . the same particles in order to remove non-specifically bound sample species from the magnetic particles.

As indicated, control of the repositioning of the magnetic field within the trapping region can be accomplished by various mechanisms. In a first embodiment, this is accomplished by moving a magnetic field producer (e.g., a permanent magnet) over one surface of the trapping region (typically outside the channel) during the passage of magnetic particles through the trapping region. FIG. 3 depicts an example of this embodiment. As shown, a permanent magnet 303 moves under a trapping region 301 during capture of magnetic particles. In the depicted embodiment, magnet 303 moves from a downstream position 305 toward an upstream position 307 during the trapping operation. It produces a magnetic field interaction volume 309 that effectively spans the height of a trapping region fluidic volume. Thus, all magnetic particles in the fluid flowing through the trapping region volume experience the force produced by the moving magnetic field producer 303.

In another embodiment, the external magnet is an electromagnet which moves along the trapping region (same as the permanent magnet) during the flow of magnetic particles into the trapping region. Optionally, the position of the magnetic field produced by the electromagnet can be controlled by other means such as mechanically moving some or all of the electromagnet's coils during the trapping period.

In another embodiment, the dynamic repositioning of the magnetic field during trapping is accomplished by sequential insertion of a series of external magnets, each of relatively small size with respect of the size of the trapping region. In one embodiment, the magnets are permanent magnets. In a specific embodiment, these permanent magnets are arranged in alternating polarities (e.g., a first magnet has its south pole oriented toward the trapping region, a second magnet has its north pole oriented toward the trapping region, a third magnet has its south pole oriented toward the trapping region, a fourth magnet has its north pole oriented toward the trapping region, etc.). FIG. 1D shows an example of such arrangement of permanent magnets.

Other details of designs and methodologies for sequential application of an external magnetic field to a trapping region are described in U.S. Provisional Patent Application No. 61/037,994, previously incorporated by reference.

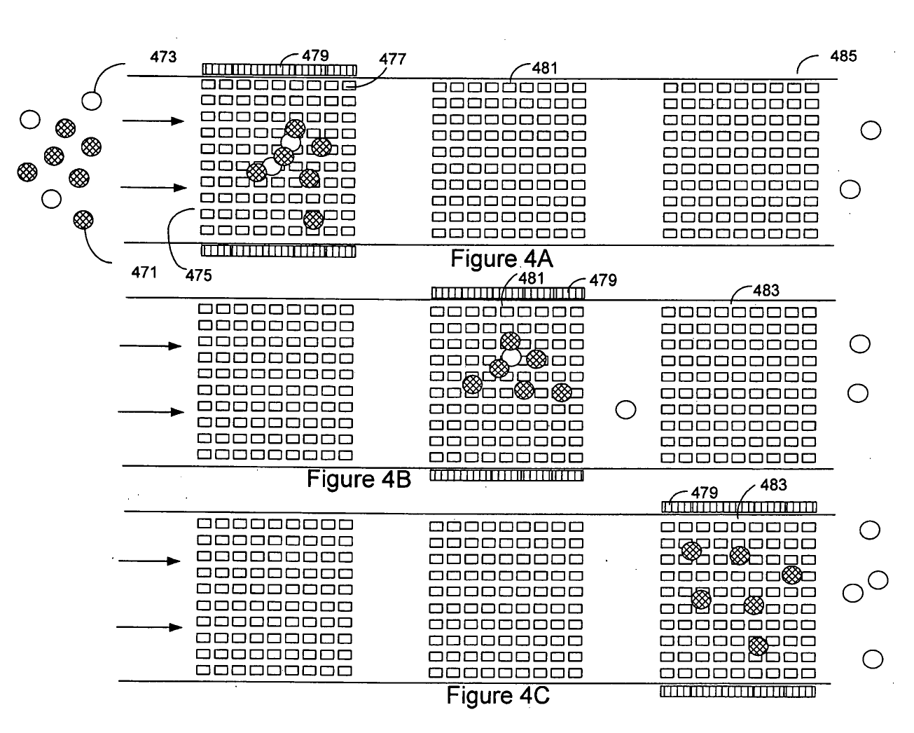

FIGS. 4A to 4C depict one example of a staged trapping system. As shown in FIG. 4A, magnetically labeled target species 471 and non-target species 473 are first flowed over a leftmost trapping region 475 of a fluidic channel 485 comprising a “soft” magnetic pattern (array of ferromagnetic structures) 477, which traps the magnetically-tagged target species 471 as well as non-specifically trapping a few non-target species 473. The non-specific trapping may be caused by factors such as physical entrapment by the target species. An external magnet 479 (which may be a collection of magnets in some embodiments) is positioned proximate trapping region 475 during this initial trapping operation. Thereafter, external magnet 479 is moved downstream to the second trapping stage 481 in FIG. 4B, allowing the trapped species to be released from the leftmost trapping stage 475. The magnetically-tagged species are trapped again in the second stage 481, and more of the non-target species are flushed out of the channel as fluid continues to flow through the stages which are aligned along a single channel. In a third trapping stage 483, shown in FIG. 4C, the last of the non-target species are flushed out of the channel, leaving only the magnetically-tagged target species on a pattern 487. External magnet 479 can now be removed to elute the target species if so desired. An advantage of using this staged system of magnetic trapping, release, and re-trapping (as opposed to a single trapping stage) is that any non-specifically bound non-target species such as cells will be more effectively removed from the magnetic traps between the consecutive trapping stages, thus enhancing the purity of the eluted target cells or other species.

In addition to the staged trapping system, varied spacing of the ferromagnetic pattern may be used. In one example, no magnetic field gradient generating structure is provided on the upstream side of the trapping region. The structure is provided only toward the downstream region. As result, the local magnetic field gradient near the lower surface of the channel is not as strong as it is further downstream where the structures reside. However, in the upstream regions without the magnetic field gradient generating structures, the magnetic field may penetrate further into the flow channel in the vertical direction. This draws the entering magnetic particles down toward the lower regions of the trapping region where they may experience—as they flow further downstream—the influence of a very strong magnetic field gradient produced by the magnetic field gradient generating structures. Very strong magnetic forces over only small distances are generated on the structures to trap the particles. The distance into the flow stream over which the magnetic forces are strong may be controlled to be the length of one monolayer of magnetic particles and labeled target particle. In some cases, bilayers and the like may be produced by self-magnetization of captured magnetic particles that then acts as capture structures for subsequent magnetic particles flowing through the trapping module.

Processing Trapped Species

In some embodiments, trapped species will be released from their associated magnetic particles in while confined to a trapping region. As mentioned, various mechanisms may be employed for this purpose. One approach involves applying a bead release agent to the trapped magnetic particles. Such agents may act by cleaving a chemical linker between the beads and the captured species or by competitively binding a linking species. Of course, other cleaving or release agents may be employed as will be understood by those of skill in the art.

In certain embodiments, fluidics input for a sample and a bead release reagent may be provided separately. During a separation process, sample is pumped from a sample well into a trapping region. Once separation process is complete, bead release reagent is pumped from the release reagent well into the trapping region. To elute the release cells, buffer can be pumped in from either of the input wells, or from a separate buffer inlets. The pumping action in all cases can be achieved using, e.g., either a gas (such as air) or liquid (such as buffered water).

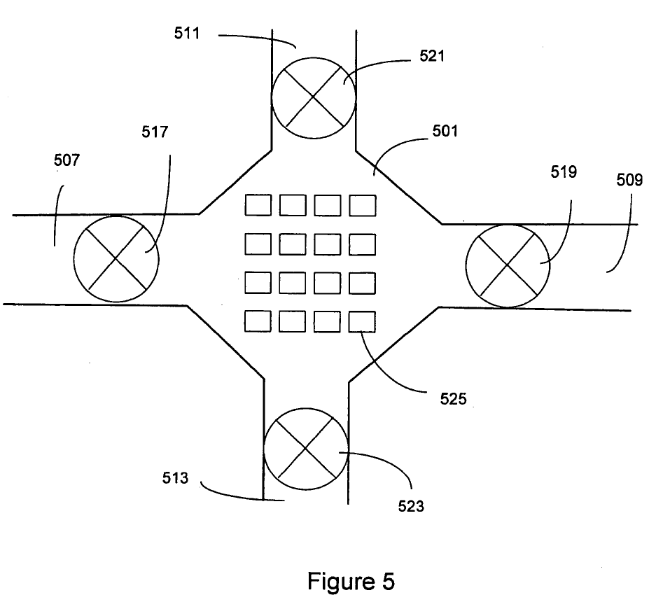

Trapped target species may be simply concentrated, purified and/or released as described. Alternatively they can be further analyzed and/or treated. This further analysis and/or treatment may occur in the trapping module or in a subsequent module after release from the trapping module. FIG. 5 shows an example structure of a magnetic trap 501 disposed in a fluidics device 505 for post-capture treatment of captured species. As shown, trap 501 includes an inlet line 507 for receiving a raw sample stream and an outlet line 509. Trap 501 also includes auxiliary lines 511 and 513 for providing one or more other reagents. Each of lines 507, 509, 511, and 513 includes its own valve 517, 519, 521, and 523, respectively. Within trap 501 are various trapping elements 525. These may be ferromagnetic elements that shape or deliver a magnetic field, etc. Although the lines and valves are shown in FIG. 5 to surround the trapping elements from four sides, the invention is not so limited. For example, other configurations include that of FIG. 1C where the inputs converge before flowing past the trapping region.

Referring to FIG. 5, while a magnetic field or other capturing stimulus is applied to the trap features 525, the particles flowing into trap 501 are captured. After a sufficient number of particles are captured (which might be indicated by simply running a sample stream through device 505 for a defined period of time), valves 517 and 519 are closed. Thereafter, in one embodiment, valves 521 and 523 are opened, and a buffer is passed from line 511, through trap 501, and out line 513. This serves to wash the captured particles. After washing for a sufficient length of time, the washed particles may be recovered by eluting (by e.g., removing an external magnetic or electrical field while the buffer continues to flow), by pipetting from trap 501, by removing a lid or cover on the trap or the entire device, etc. Regarding the last option, note that in some embodiments the devices are disposable and can be designed so that the top portion or a cover is easily removed by, e.g., peeling.

In another embodiment, the particles that have been captured and washed in the trap as described above are exposed to one or more markers (e.g., labeled antibodies) for target cells or other target species in the sample. Certain tumor cells to be detected, for example, express two or more specific surface antigens. This combination of antigens occurs only in very unique tumors. To detect the presence of such cells bound to magnetic particles, valves 517 and 523 may be closed and valve 521 opened after capture in trap 501 is complete. Then a first label is flowed into trap 501 via line 511 and out via line 509. Some of the label may bind to immobilized cells in trap 501. Thereafter, valve 521 is closed and valve 523 is opened and a second label enters trap 501 via line 513. After label flows through the trap for a sufficient length of time, the captured particles/cells may be washed as described above. Thereafter, the particles/cells can be removed from trap 501 for further analysis or they may be analyzed in situ. For example, the contents of trap 501 may be scanned with probe beams at excitation for the first and second labels if such labels or fluorophores for example. Emitted light is then detected at frequencies characteristic of the first and second labels. In certain embodiments, individual cells or particles are imaged to characterize the contents of trap 501 and thereby determine the presence (or quantity) of the target tumor cells. Of course various target components other than tumor cells may be detected. Examples include pathogens such as certain bacteria or viruses.

In another embodiment, nucleic acid from a sample enters trap 501 via line 507 and is captured by an appropriate mechanism (examples set forth below). Subsequently, valve 517 is closed and PCR reagents (nucleotides, polymerase, and primers in appropriate buffers) enter trap 501 via lines 511 and 513. Thereafter all valves (517, 519, 521, and 523) are closed and an appropriate PCR thermal cycling program is performed on trap 501. The thermal cycling continues until an appropriate level of amplification is achieved. Subsequently in situ detection of amplified target nucleic acid can be performed for, e.g., genotyping. Alternatively, the detection can be accomplished downstream of trap 501 in, e.g., a separate chamber which might contain a nucleic acid microarray or an electrophoresis medium. In another embodiment, real time PCR can be conducted in trap 501 by introducing, e.g., an appropriately labeled intercalation probe or donor-quencher probe for the target sequence. The probe could be introduced with the other PCR reagents (primers, polymerase, and nucleotides for example) via line 511 or 513. In situ real time PCR is appropriate for analyses in which expression levels are being analyzed. In either real time PCR or end point PCR, detection of amplified sequences can, in some embodiments, be performed in trap 501 by using appropriate detection apparatus such as a fluorescent microscope focused on regions of the trap.

For amplification reactions, the capture elements 525 capture and confine the nucleic acid sample to reaction chamber 501. Thereafter, the flow through line 507 is shut off and a lysing agent (e.g., a salt or detergent) is delivered to chamber 501 via, e.g., line 511 or 513. The lysing agent may be delivered in a plug of solution and allowed to diffuse throughout chamber 101, where it lyses the immobilized cells in due course. This allows the cellular genetic material to be extracted for subsequent amplification. In certain embodiments, the lysing agent may be delivered together with PCR reagents so that after a sufficient period of time has elapsed to allow the lying agent to lyse the cells and remove the nucleic acid, a thermal cycling program can be initiated and the target nucleic acid detected.

In other embodiments, sample nucleic acid is provided in a raw sample and coupled to magnetic particles containing appropriate hybridization sequences. The magnetic particles are then sorted and immobilized in trap 501. After PCR reagents are delivered to chamber 501 and all valves are closed, PCR can proceed via thermal cycling. During the initial temperature excursion, the captured sample nucleic acid is released from the magnetic particles.

The nucleic acid amplification technique described here is a polymerase chain reaction (PCR). However, in certain embodiments, non-PCR amplification techniques may be employed such as various isothermal nucleic acid amplification techniques; e.g., real-time strand displacement amplification (SDA), rolling-circle amplification (RCA) and multiple-displacement amplification (MDA). Each of these can be performed in a trap such as chamber 501 shown in FIG. 5.

Example Magnetic Trapping Structures

Most fundamentally, a trapping station is defined by the boundaries of a region or channel in a fluidics device. Fluid flows through the trapping station and encounters a magnetic field generated by one or more external magnets proximate the trapping station. In addition, a trapping station may optionally employ a magnetic field gradient generating structure (MFGs). MFG elements (e.g., strips, pins, dots, grids, random arrangements, etc.) shape the external magnet field to produce a locally high magnetic field gradient in the trapping station.

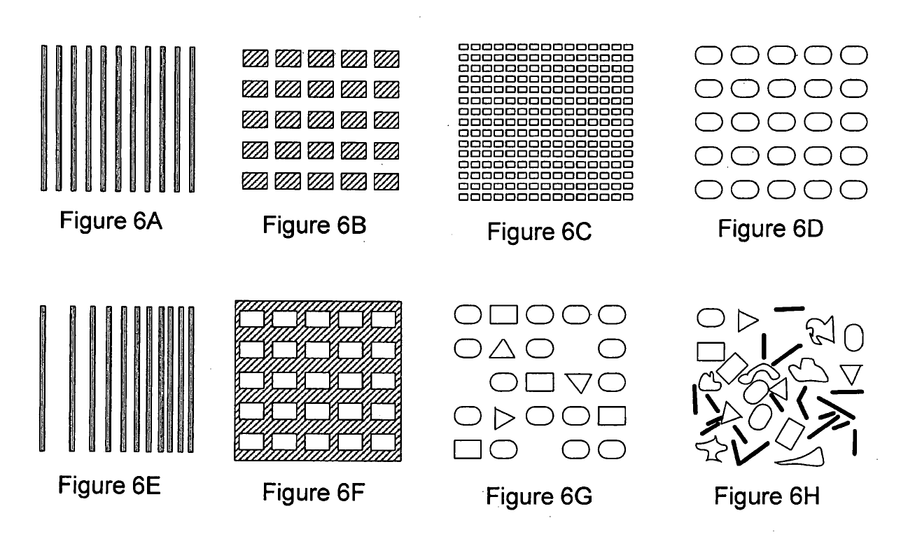

FIG. 6 depicts examples of different types of ferromagnetic MFG structures that may be employed with magnetic trapping stations this invention. Eight different ferromagnetic element patterns are shown in the figure. These are employed to shape a magnetic field gradient originating from an external source of a magnetic field (not shown). As shown, the ferromagnetic structures are provided in an organized or a random pattern, such as parallel lines, an orthogonal grid, and rectangular arrays of regular or irregular geometric shapes. The structures may be regular or reticulated as shown.

Generally, the features or elements in these patterns may be made from various materials having permeabilities that are significantly different from that of the fluid medium in the device (e.g., the buffer). As indicated, the elements may be made from a ferromagnetic material. In a specific embodiment, the patterns are defined by nickel features on a glass or polymer substrate.

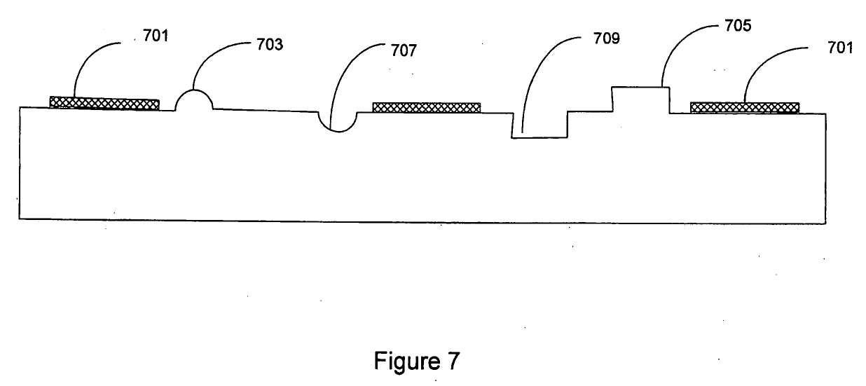

In alternative embodiments, the MFG structures are combined with other types of capture structures such as electrodes, specific binding moieties (e.g., regions of nucleotide probes or antibodies), physical protrusions or indentations, etc. FIG. 7 presents examples of non-magnetic capture features that are fabricated among a soft-magnetic (e.g., nickel) pattern 701. The patterns may be positive or negative surface features to facilitate laminar mixing of the fluid over the nickel structures, causing enhanced magnetic trapping. FIG. 7 shows the magnetic field gradient generating structures 701 with non-magnetic capture features. Positive features include round bump 703 and square bump 705. Negative features include divot 707 and square hole 709. Each of these features may be used for its geometrical effect on the flow mixing or be coated with a substance that aids a reaction or flow.

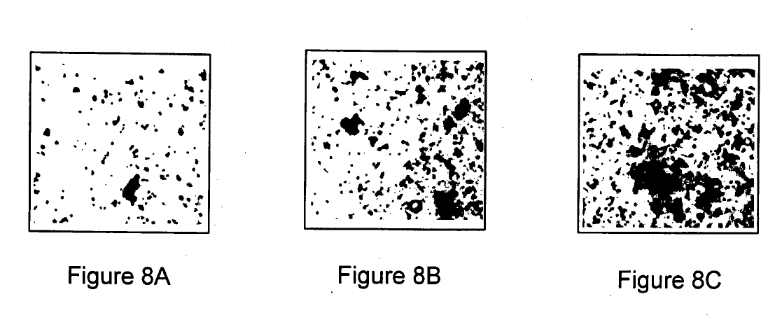

Other types of MFG structures comprise ferromagnetic materials that do not form well-defined shapes or regular features. Instead, the structures form randomly placed features such as randomly dispersed powder, filings, granules, etc. These structures are affixed to one or more walls of the trapping station adhesives, pressure bonding, etc. FIG. 8A to 8C show examples of random array of ferromagnetic structures from left to right: 5% (FIG. 8A), 10% (FIG. 8B) and 30% (FIG. 8C) nickel powder in an epoxy resin. Such structures have found to be effective MFG elements in magnetic trapping stations.

In an alternative embodiment, the trapping station contains no MFG structures. Instead, magnetic capture is based solely on the strength of the external magnetic field, without the aid of a field shaping element such as MFG structures.

Fluidics and Sorting Chamber Design

While some embodiments of this invention are implemented in micro-scale microfluidic systems, it should be understood that methods, apparatus, and systems of this invention are not limited to microfluidic systems. Typical sizes of larger trapping chambers range between about 1 and 100 millimeters in length (in the direction of flow), between about 1 and 100 millimeters in width and between about 1 micrometer and 10 millimeters depth (although typically about 1 millimeter or less). The depth and width together define the cross section through which fluid flows. The depth represents the dimension in the direction that the magnetic field penetrates into the channel, typically a direction pointed away from the position of the external magnet. In certain embodiments, the chambers have an aspect ratio (length to width) that is greater than 1, e.g., about 2 to 8.

In general, the applied magnetic field should be sufficiently great to capture or trap magnetic particles flowing in a fluid medium. Those of skill in the art will recognize that the applied magnetic force must be significantly greater than the hydrodynamic force exerted on the particles by the flowing fluid. This may limit the depth dimension of the trapping station.

In certain embodiments, the integrated fluidics systems are microfluidic systems. Microfluidic systems may be characterized by devices that have at least one “micro” channel. Such channels may have at least one cross-sectional dimension on the order of a millimeter or smaller (e.g., less than or equal to about 1 millimeter). Obviously for certain applications, this dimension may be adjusted; in some embodiments the at least one cross-sectional dimension is about 500 micrometers or less. In some embodiments, as applications permit, a cross-sectional dimension is about 100 micrometers or less (or even about 10 micrometers or less—sometimes even about 1 micrometer or less). A cross-sectional dimension is one that is generally perpendicular to the direction of centerline flow, although it should be understood that when encountering flow through elbows or other features that tend to change flow direction, the cross-sectional dimension in play need not be strictly perpendicular to flow. Often a micro-channel will have two or more cross-sectional dimensions such as the height and width of a rectangular cross-section or the major and minor axes of an elliptical cross-section. Either of these dimensions may be compared against sizes presented here. Note that micro-channels employed in this invention may have two dimensions that are grossly disproportionate—e.g., a rectangular cross-section having a height of about 100-200 micrometers and a width on the order or a centimeter or more. Of course, certain devices may employ channels in which the two or more axes are very similar or even identical in size (e.g., channels having a square or circular cross-section).

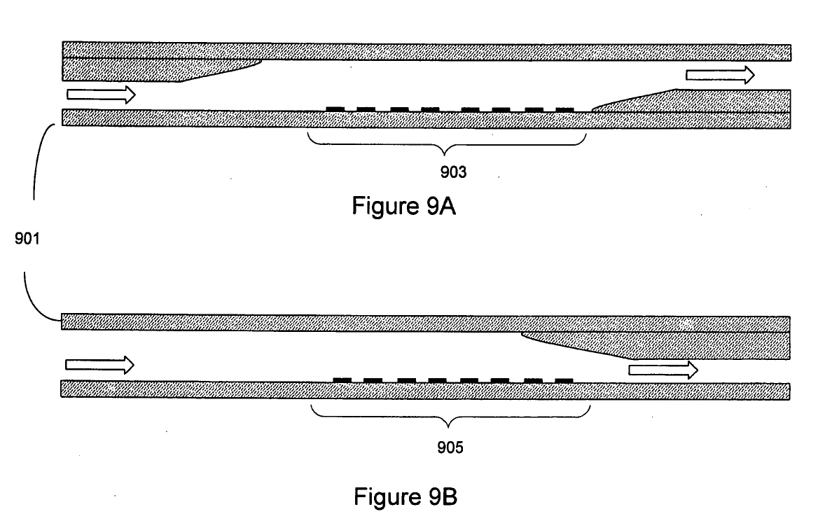

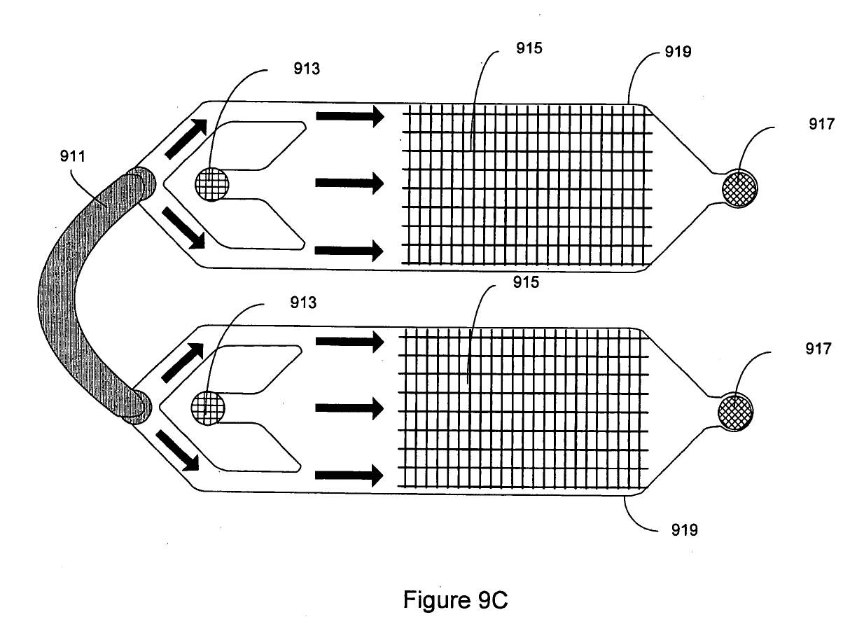

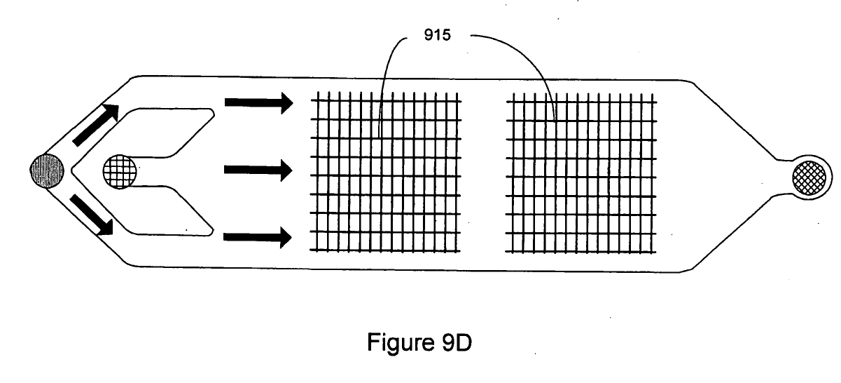

In some embodiments, the fluidic channel may have a larger depth in the trapping region to slow down the particle velocity for improved magnetic sedimentation and capture. FIGS. 9A and 9B show side views of example fluidic channels 901 that include larger depth trapping regions (903 and 905) in comparison to the inlet and/or outlet portions of the channel. In one example (FIG. 9A) the channel depth increases in the trapping region 903 (along the direction of flow from the inlet to the trapping region) and decreases thereafter. In another example (FIG. 9B), the channel depth decreases after (downstream from) the trapping region 905. Typical ratios of depth in trapping region to depth in the inlet or outlet channels range between about 1 and 5 (typically about 2). The shape of the transition region between the trapping region and inlet or outlet channels has a smooth curved channel to prevent flow recirculation or physical trapping at the corners. In certain embodiments, the fluidic channel may have the same depth, but a larger width near the inlet channel and narrower width near the outlet channel.

In certain embodiments as shown in FIG. 9C, the sorting device may comprise two or more parallel channels 919 with a common buffer manifold 911 for multiple concurrent sample separations and/or increased throughput. In both channels 919, flow from a sample inlet 913 combines with buffer from a manifold 911 and flows through a magnetic field gradient generating structure 915 in a trapping region to an outlet 917.

In still other embodiments, the fluidic channel may include more than one trapping region to allow a capture and release protocol. FIG. 9D depicts a sorting chamber configured to perform a capture and release protocol described above in reference to FIGS. 4A to 4C. As shown in FIG. 9D, two or more trapping regions 915 may be included in the fluidic channel. Each of these trapping regions may have a corresponding external magnetic source that allows a magnetic field to be applied independently to each region. The trapping regions may share one external magnetic source. In embodiments where two external magnetic sources are used, these sources may be configured to provide a time-varying magnetic field independently or together. One way to vary the magnetic field is to move the external sources relative to the fluidic channel. The external magnetic source may move in a direction parallel or perpendicular to the fluid flow to generate the desired magnetic field variations.

Often a controller will be employed to coordinate the operations of the various systems or sub-systems employed in the overall microfluidic system. Such controller will be designed or configured to direct the sample through a microfluidic flow passage. It may also control other features and actions of the system such as the strength and orientation of a magnetic field applied to fluid flowing through the microfluidic device, control of fluid flow conditions within the microfluidic device by actuating valves and other flow control mechanisms, mixing of magnetic particles and sample components in an attachment system, generating the sample (e.g., a library in a library generation system), and directing fluids from one system or device to another. The controller may include one or more processors and operate under the control of software and/or hardware instructions.

Integration

Examples of operational modules that may be integrated with magnetic trapping sorters in fluidics devices include (a) additional enrichment modules such as fluorescence activated cell sorters and washing modules, (b) reaction modules such as sample amplification (e.g., PCR) modules, restriction enzyme reaction modules, nucleic acid sequencing modules, target labeling modules, chromatin immunoprecipitation modules, crosslinking modules, and even cell culture modules, (c) detection modules such as microarrays of nucleic acids, antibodies or other highly specific binding agents, and fluorescent molecular recognition modules, and (d) lysis modules for lysing cells, disrupting viral protein coats, or otherwise releasing components of small living systems. Each of these modules may be provided before or after the magnetic sorter. There may be multiple identical or different types of operational modules integrated with a magnetic sorter in a single fluidics system. Further, one or more magnetic sorters may be arranged in parallel or series with respect to various other operational modules. Some of these operational modules may be designed or configured as traps in which target species in a sample are held stationary or generally constrained in particular volume.

As should be apparent from the above examples of modules, operations that may be performed on target and/or non-target species in modules of integrated fluidics devices include sorting, coupling to magnetic particles (sometimes referred to herein as “labeling”), binding, washing, trapping, amplifying, removing unwanted species, precipitating, cleaving, diluting, ligating, sequencing, synthesis, labeling (e.g., staining cells), cross-linking, culturing, detecting, imaging, quantifying, lysing, etc.

Specific examples of biochemical operations that may be performed in the magnetic sorting modules of integrated fluidic devices include synthesis and/or screening of plasmids, aptamers, proteins, and peptides; evaluating enzyme activity; and derivatizing proteins and carbohydrates. A broad spectrum of biochemical and electrophysiological assays may also be performed, including: (1) genomic analysis (sequencing, hybridization), PCR and/or other detection and amplification schemes for DNA, and RNA oligomers; (2) gene expression; (3) enzymatic activity assays; (4) receptor binding assays; and (5) ELISA assays. The foregoing assays may be performed in a variety of formats, such as: homogeneous, bead-based, and surface bound formats. Furthermore, devices as described herein may be utilized to perform continuous production of biomolecules using specified enzymes or catalysts, and production and delivery of biomolecules or molecules active in biological systems such as a therapeutic agents. Microfluidic devices as described herein may also be used to perform combinatorial syntheses of peptides, proteins, and DNA and RNA oligomers as currently performed in macrofluidic volumes.

Examples of Reactors and Lysis Modules in Fluidics Systems

An integrated microfluidic device in accordance of the present invention may employ various reactors with various features. The exact feature depends on the type of reaction and may include a thermal management system, micromixer, catalyst structure and sensing system. A thermal management system may include heaters, temperature sensors, and micro heat exchangers. All these components may be integrated to precisely control temperatures. Such temperatures control is crucial for PCR for DNA amplification.

Micromixers may be used for mixing two solutions. An example micromixer involves pressurizing one fluid through a hole or slit in a separating material to induce diffusion between two fluids. The separating material is hydrophobic and separates two adjacent chambers. The hydrophobicity of the interface permits both chambers to be filled with fluid without mixing. A pressure gradient can then be applied to force fluid through the hole in the hydrophobic layer to induce diffusion that makes a reaction possible.

Catalyst structures are used to accelerate a chemical reaction (e.g., cross-linking or sequencing). In microreactors, the catalyst may be fixed, e.g., fixed beads, wires, thin films or a porous surface, or added. Thin films and porous surface catalysts may be easily integrated in the fabrication of microreactors. A sensing system may employ chemical microsensors, or biosensors, for example. Optical sensing may also be performed externally through a glass or plastic surface.

Contents of a biological cell are often subject of manipulation and analysis. Before the content of a cell may be analyzed, the cell is lysed so that the components of the cell can be separated. The methods of cell disruption used to release the biological molecules in a cell and in a virus include, e.g., electric field, enzyme, sonication, and using a detergent. Mechanical forces may also be used to shear and burst cell walls.

The cell lysis may be performed by subjecting the cells trapped in a reaction chamber to pulses of high electric field strength, typically in the range of about 1 kV/cm to 10 kV/cm. The use of enzymatic methods to remove cell walls is well-established for preparing cells for disruption, or for preparation of protoplasts (cells without cell walls) for other uses such as introducing cloned DNA or subcellular organelle isolation. The enzymes are generally commercially available and, in most cases, were originally isolated from biological sources (e.g. snail gut for yeast or lysozyme from hen egg white). The enzymes commonly used include lysozyme, lysostaphin, zymolase, cellulase, mutanolysin, glycanases, proteases, mannase etc.

In addition to potential problems with the enzyme stability, the susceptibility of the cells to the enzyme can be dependent on the state of the cells. For example, yeast cells grown to maximum density (stationary phase) possess cell walls that are difficult to remove whereas midlog growth phase cells are much more susceptible to enzymatic removal of the cell wall. If an enzyme is used, it may have to be sorted and removed from the desired material before further analysis.

Sonication uses a high-frequency wave that mechanically burse the cell walls. Ultrasound at typically 20-50 kHz is applied to the sample via a metal probe that oscillates with high frequency. The probe is placed into the cell-containing sample and the high-frequency oscillation causes a localized high pressure region resulting in cavitation and impaction, ultimately breaking open the cells. Cell disruption is available in smaller samples (including multiple samples under 200 μL in microplate wells) and with an increased ability to control ultrasonication parameters.

A mechanical method for cell disruption uses glass or ceramic beads and a high level of agitation to shear and burst cell walls. This process works for easily disrupted cells, is inexpensive, but has integration issues for the micrfluidic device. In one embodiment, beads are used in a closed chamber holding the sample and are agitated with an electric motor. In other embodiments, high pressure is applied to fluid containing the cell samples while forcing the fluid to flow through a very narrow channel. Shear between the cell and channel walls under such conditions would disrupt the cell.

Detergent-based cell lysis is an alternative to physical disruption of cell membranes, although it is sometimes used in conjunction with homogenization and mechanical grinding. Detergents disrupt the lipid barrier surrounding cells by disrupting lipid:lipid, lipid:protein and protein:protein interactions. The ideal detergent for cell lysis depends on cell type and source and on the downstream applications following cell lysis. Animal cells, bacteria and yeast all have differing requirements for optimal lysis due to the presence or absence of a cell wall. Because of the dense and complex nature of animal tissues, they require both detergent and mechanical lysis to effectively lyse cells.

In general, nonionic and zwitterionic detergents are milder, resulting in less protein denaturation upon cell lysis, than ionic detergents and are used to disrupt cells when it is critical to maintain protein function or interactions. CHAPS, a zwitterionic detergent, and the Triton X series of nonionic detergents are commonly used for these purposes. In contrast, ionic detergents are strong solubilizing agents and tend to denature proteins, thereby destroying protein activity and function. SDS, and ionic detergent that binds to and denatures proteins, is used extensively for studies assessing protein levels by gel electrophoresis and western blotting.

Examples of Detectors in Integrated Flow Systems

In various applications envisaged for integrated Microsystems it will be necessary to quantify the material present in a channel at one or more positions similar to conventional laboratory measurement processes. Techniques typically utilized for quantification include, but are not limited to, optical absorbance, refractive index changes, fluorescence emission, chemiluminescence, various forms of Raman spectroscopy, electrical conductometric measurements, impedance measurements (e.g., impedance cytometry) electrochemical amperiometric measurements, and computer assisted optical imaging and counting.

Optical absorbance is determined by measuring the light intensity as it passes through the material to be quantified. The level of attenuation through the material indicates the amount of material, if the optical properties are known. Alternative approaches include photo acoustic and photo thermal techniques user laser technology. A microfluidic device may include optical wave guides and solid-state optical sources, e.g., LEDs and diode lasers, to minimize the size of the integrated device.

Similar to optical absorbance is refractive index. Refractive index detectors quantify the refractive index of the sample material, which may be correlated to a quantity of the sample; however, this technique is less sensitive than optical absorption. Laser based and fluorescence emission based implementations of refractive index detection are also available and are more sensitive. Laser and fluorescence based approaches may be used with very small volumes and is suitable for integration in a microfluidic device. The excitation source for ultrasensitive measurements may be a laser source, rare gas discharge lamps, and light emitting diodes (LEDs). The fluorescence emission can be detected by a photomultiplier tube, photodiode or other light sensor.

An array detector such as a charge coupled device (CCD) detector can be used to image an analyte spatial distribution. A computer program may be used to analyze the image. As discussed above, one feature of the present invention is the ability to form a monolayer of target particles, presenting a uniform depth of focus. A computer program may analyze the image by counting particles of a certain size or color and thus quantify the sample.

Raman spectroscopy relies on inelastic scattering, or Raman scattering of monochromatic light from interactions with phonons or other excitations in the system that result in the energy of the laser photons being shifted up or down. The shift in energy gives information about the phonon modes in the system such as molecular vibrational information. Various types of Raman spectroscopy with enhanced sensitivity are available for microscopic volumes.

Electrical or electrochemical detection approaches may be easily integrated onto a microfludic device and may be more sensitive than other approaches. Electrical or electrochemical approaches include measuring conductivity in an ionized analyte, measuring current through an electrode at a given electrical potential by reducing or oxidizing a molecule at the electrode. The number of electrons measured is equal to the number of molecules present. Electrodes could also be used to initiate a chemiluminescence detection process where a molecule transfers its energy from an oxidation-reduction process to an analyte molecule that emits a detectible photon.

Detection processes that require adding or mixing one or more reagents can be easily integrated onto a microfluidic system. Derivatization reactions are commonly used in biochemical assays. For example, amino acids, peptides and proteins are commonly labeled with dansylating reagents or o-phthaldialdehyde to produce fluorescent molecules that are easily detectable. Alternatively, an enzyme could by used as a labeling molecule and reagents, including substrate, could be added to provide an enzyme amplified detection scheme, i.e., the enzyme produces a detectable product. A third example of a detection method that could benefit from integrated mixing methods is chemiluminescence detection. In these types of detection scenarios, a reagent and a catalyst are mixed with an appropriate target molecule to produce an excited state molecule that emits a detectable photon.

Many of these detection and quantification methods involve some altering of the sample. In some cases, a reaction occurs such as oxidation or reduction. In other cases, the light flux has the potential to degrade the sample. Depending on the amount of sample to be quantified and the necessity of preserving the sample for other processing, e.g., reacting or even archiving, these methods may be selected singly or in combination to yield information about the sample.

### CONCLUSION

A number of embodiments of the invention have been described. Nevertheless, it will be understood that various modifications may be made without departing from the spirit and scope of the invention. For example, the above description has been focused on biological applications and in particular biological cell detection and trapping, but it should also be noted that the same principles apply to other particles, such as inorganic or non-biological organic materials. Thus, the apparatus and methods described above can also be used for non-biological substances in liquids. Accordingly, other embodiments are within the scope of the following claims.

What is claimed is:

1. A microfluidic sorting device for sorting a sample into substantially target and substantially non-target species, the device comprising:
(a) a trapping module comprising a channel having two opposing walls, a magnetic field gradient generating structure on only one of the opposing walls for exerting a magnetic force on a sample to capture, at least temporarily, magnetic particles in the sample; and
(b) a pre-processing and/or a post-processing station integrated on the microfluidic sorting device with trapping module.

2. The microfluidic sorting device of claim 1, wherein the opposing wall having the magnetic field gradient generating structure is on an opaque wall.

3. The microfluidic sorting device of claim 1, further comprising an external source on one side of the trapping module for generating a magnetic field.

4. The microfluidic sorting device of claim 3, wherein the external source is located on the same side of the trapping module as the magnetic field gradient generating structure.

5. The microfluidic sorting device of claims 3, further comprising a mechanism for moving the external source of the magnetic field with respect to the trapping module.

6. The microfluidic sorting device of claims 3, wherein the external source of the magnetic field comprises one or more permanent magnets.

7. The microfluidic sorting device of claim 6, wherein the permanent magnets alternate in polarity.

8. The microfluidic sorting device of claim 3, wherein the external source of the magnetic field comprises one or more electromagnets.

9. The microfluidic sorting device of claim 1, wherein the magnetic field gradient generating structure comprises a ferromagnetic structure laid out in a pattern on its opposing wall.

10. The microfluidic sorting device of claim 9, wherein the pattern is selected from the group consisting of parallel lines, an orthogonal grid, a rectangular array of regular or irregular geometric shapes, and combinations thereof.

11. The microfluidic sorting device of claim 1, wherein the magnetic field gradient generating structure comprises a random array of ferromagnetic structures.

12. The microfluidic sorting device of claims 9, wherein the ferromagnetic structure material is nickel, vanadium permedur, or permalloy.

13. The microfluidic sorting device of claim 1, wherein the channel has a larger depth in the trapping region.

14. The microfluidic sorting device of claim 1, wherein the trapping module comprises a staged trapping system with two or more trapping regions.

15. The microfluidic sorting device of claim 1, further comprising a second parallel, independent trapping module; and a common buffer manifold connecting the trapping modules.

16. The microfluidic sorting device of claim 1, wherein the pre-processing station comprises a labeling station for labeling a species in the sample with magnetic particles having an affinity for the labeled species.

17. The microfluidic sorting device of claim 1, wherein the post-processing station comprises a detection station for detecting the target species.

18. The microfluidic sorting device of claim 1, wherein at least one of the pre-processing station or the post-processing station comprise (a) an enrichment module for increasing a concentration of a target species in a sample passing through the sorting device, (b) a reaction module, (c) a detection module, and (d) a lysis module for lysing cells, disrupting viral protein coats, or otherwise releasing components of small living systems.

19. The microfluidic sorting device of claim 1, wherein the trapping module is designed or configured to perform an operation selected from the group consisting of (1) genomic analysis; (2) a detection and/or amplification scheme for DNA or RNA oligomers; (3) gene expression; (4) enzymatic activity assays; (5) receptor binding assays; and (6) ELISA assays.

20. A method for sorting a sample in a microfluidic sorting device that includes a trapping module and a magnetic field gradient generating structure on only one of two opposing walls of the trapping module, the method comprising:
flowing a sample into the trapping module, said sample comprising a plurality magnetic particles with molecular recognition elements thereon, a target species, and a non-target species;
generating a magnetic field gradient in the trapping module by exerting an external magnetic field from only one side of the module to the magnetic field gradient generating structure; and
trapping magnetic particles in the trapping module proximate to the magnetic field gradient generating structure on one wall of the trapping module.

21. The method for sorting a sample of claim 20, further comprising:
flowing a buffer concurrently with the sample into the trapping module.

22. The method for sorting a sample of claims 21, wherein the buffer is continuously flowed into the trapping module during every operation.

23. The method for sorting a sample of claims 20, further comprising:
moving an external source of the magnetic field with respect to the trapping module while the magnetic particles flow through the module in the fluid medium to thereby trap magnetic particles in a substantially uniform fashion.

24. The method for sorting a sample of claims 20, further comprising:
releasing the magnetic particles from one section of the magnetic field gradient generating structure to release any trapped non-magnetic particles; and
trapping the magnetic particles in another section of the magnetic field gradient generating structure.

25. The method for sorting a sample of claim 20, further comprising:
labeling the target species in the sample with magnetic particles having an affinity for the target species.

26. The method for sorting a sample of claim 20, further comprising:
labeling the non-target species in the sample with magnetic particles having an affinity for the non-target species.

27. The method for sorting a sample of claim 20, further comprising:
detecting the target species in a microarray.

28. The method for sorting a sample of claim 20, further comprising:
lysing the target species.

29. The method for sorting a sample of claim 20, further comprising:
reacting the target species to amplify, sequence, hybridize, label, crosslink or culture the target species.

30. The method for sorting a sample of claim 20, further comprising:
imaging the trapped target species.

31. A microfluidic sorting device for sorting a sample into substantially target and substantially non-target species, the device comprising:
(a) a trapping module comprising a channel having two opposing walls and no magnetic field gradient generating structure;
(b) a pre-processing and/or a post-processing station integrated on the microfluidic sorting device with trapping module;
(c) an external source on one side of the trapping module for generating a magnetic field; and
(d) a mechanism to vary the magnetic field over time.
